(19) United States
(12) Patent Application Publication
(10) Pub. No.: US 2014/0271364 A1
(43) Pub. Date: Sep. 18, 2014

(54) ELECTRO-IMMUNO SENSING DEVICE

(71) Applicant: NATIONAL TAIWAN UNIVERSITY, Taipei City (TW)

(72) Inventors: Shiming Lin, Taipei City (TW); Luan-Yin Chang, Taipei City (TW); Bor-Ching Sheu, Taipei City (TW)

(73) Assignee: NATIONAL TAIWAN UNIVERSITY, Taipei City (TW)

(21) Appl. No.: 14/184,750

(22) Filed: Feb. 20, 2014

Related U.S. Application Data
(60) Provisional application No. 61/799,915, filed on Mar. 15, 2013.

(51) Int. Cl. G01N 33/543; G01N 33/72

(52) U.S. Cl. CPC G01N 33/5438; G01N 33/721
USPC 422/69

(57) ABSTRACT

An electro-immuno sensor includes a signal generator, a sensing chip and a sensing circuit. The signal generator is arranged for generating an electronic signal having a predetermined frequency and a waveform. The sensing chip is disposed in an electrical field that generates the electronic signal, and arranged for capturing target biomolecules in a solution using antibodies. The sensing circuit has a first electrode and a second electrode in contact with the solution, and arranged for measuring an electrical characteristic of a first state and the electrical characteristic of a second state between the first and second electrodes, due to the capture of the target biomolecules, and determining the concentration of the target biomolecules according to the variation of the electrical characteristic.

### CROSS-REFERENCE TO RELATED APPLICATION

This application claims the priority of U.S. patent application No. 61/799,915, filed on Feb. 20, 2013, which is incorporated herewith by reference.

### BACKGROUND OF THE INVENTION

1. Field of the Invention

The present invention generally relates to a device for quantitatively measuring the concentrations of specific biomolecules in blood samples, in particular, to a device that measures the concentrations of hemoglobin and glycosylated hemoglobin and then determines the percentage of glycosylated hemoglobin in blood samples based on capacitance or resistance measured across a container containing a blood sample.

2. Description of the Prior Art

Conventional devices, such as the Hemocue® Analyzer by Sweden and DCA Vantage® Analyzer by Siemens, for quantitatively measuring the concentration of hemoglobin and determining the percentage of glycosylated hemoglobin in blood, respectively, may include a spectrophotometer that analyzes the intensity of the light transmitted through a microcuvette or a cartridge containing a blood sample. However, the spectrophotometer is expensive and requires some expertise to use.

Therefore, there is a need to provide a device that can quantitatively determine the concentration of hemoglobin and glycosylated hemoglobin in blood samples by using a simple circuit to measure electrical characteristics across a container containing a blood sample.

### BRIEF SUMMARY OF THE INVENTION

In accordance with exemplary embodiments of the present invention, an electro-immuno sensing device using electrical characteristics across a container containing a blood sample to determine the concentration of hemoglobin and glycosylated hemoglobin therein is proposed to solve the above-mentioned problem.

According to one aspect of the present invention, an exemplary electro-immuno sensing device is disclosed. The exemplary electro-immuno sensing device includes a signal generator, a sensing chip and a sensing circuit. The signal generator is arranged for generating an electronic signal having a predetermined frequency and a waveforms. The sensing chip is disposed in an electrical field that generates the electronic signal, and arranged for capturing target biomolecules in a solution using antibodies. The sensing circuit has a first electrode and a second electrode in contact with the solution, and arranged for measuring an electrical characteristic of a first state and the electrical characteristic of a second state between the first and second electrodes, due to the capture of the target biomolecules, and determining the concentration of the target biomolecules according to the variation of the electrical characteristic.

Additional features and advantages of the present invention will be set forth in portion in the description which follows, and in portion will be obvious from the description, or may be learned by practice of the invention. The features and advantages of the invention will be realized and attained by means of the elements and combinations particularly pointed out in the appended claims.

It is to be understood that both the foregoing general description and the following detailed description are exemplary and explanatory only and are not restrictive of the invention as claimed.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, examples are shown in the drawings. It should be understood, however, that the drawings are not to scale, and that the invention is not limited to the precise arrangements and instrumentalities shown in the examples.

In the drawings:

FIG. 1 illustrates a block diagram of an electro-immuno sensing device in accordance with an embodiment of the present invention;

FIG. 2 illustrates a schematic diagram of an electro-immuno sensing device 20 in accordance with a first embodiment of the present invention;

FIG. 3 illustrates a schematic diagram of an electro-immuno sensing device 30 in accordance with a second embodiment of the present invention;

FIG. 4 illustrates a schematic diagram of an electro-immuno sensing device 40 in accordance with a third embodiment of the present invention;

FIG. 5 illustrates a schematic diagram of an electro-immuno sensing device 50 in accordance with a fourth embodiment of the present invention;

FIG. 6 illustrates a schematic diagram of an electro-immuno sensing device 60 in accordance with a fifth embodiment of the present invention;

FIG. 7 illustrates a schematic diagram of an electro-immuno sensing device 70 in accordance with a sixth embodiment of the present invention;

FIG. 8 illustrates a schematic diagram of an electro-immuno sensing device 80 in accordance with a seventh embodiment of the present invention;

FIG. 9 illustrates a schematic diagram of an electro-immuno sensing device 90 in accordance with a eighth embodiment of the present invention;

FIG. 10 illustrates a graph of parallel resistance vs. hemoglobin concentration;

FIG. 11 illustrates a graph of series resistance vs. hemoglobin concentration;

FIG. 12 illustrates a graph of series capacitance vs. hemoglobin concentration; and

FIG. 13 illustrates a graph of series resistance vs. glycosylated hemoglobin concentration.

### DETAILED DESCRIPTION OF THE INVENTION

Reference will now be made in detail to the present examples of the invention illustrated in the accompanying drawings. Wherever possible, the same reference numbers will be used throughout the drawings to refer to the same or like portions. It should be noted that the drawings are in greatly simplified form and are not to precise scale.

Certain terms are used throughout the description and following claims to refer to particular components. As one skilled in the art will appreciate, manufacturers may refer to a component by different names. This document does not intend to distinguish between components that differ in name but not function. In the following description and in the claims, the terms “include” and “comprise” are used in an open-ended fashion, and thus should be interpreted to mean “include, but not limited to . . . ”. Also, the term “couple” is intended to mean either an indirect or direct electrical connection. Accordingly, if one device is electrically connected to another device, that connection may be through a direct electrical connection, or through an indirect electrical connection via other devices and connections.

FIG. 1 illustrates a function block diagram of an electro-immuno sensing device 10 in accordance with an embodiment of the present invention. The electro-immuno sensing device 10 includes a sensing chip 100 and an electronic test unit 200. The sensing chip 100 is configured to be electrically coupled with the electronic test unit 200. The electronic test unit 200 includes a signal generator 210 and a sensing circuit 220. The signal generator 210 is configured to generate an electronic signal SIG having a predetermined waveform and frequency that travels from a first location on the sensing chip 100 to a second location on the sensing chip 100. That is, the sensing chip 100 is in the electrical field that generates the electronic signal SIG. The waveform of the signal SIG may be a sine wave, a square wave, a triangular wave or a sawtooth wave, and the frequency of the signal SIG between 0.5 hertz to 2 megahertz. The sensing circuit 220 measures an electrical characteristic, such as capacitance, impedance, inductance or phase angle between the two locations over a predetermined period of time, and determines the concentration of target biomolecules (e.g., hemoglobin, or glycosylated hemoglobin) in a test sample 900. Please note that, in the embodiments, the biomolecules is hemoglobin or glycosylated hemoglobin. However, it is for illustrative purpose only, and not meant to be a limitation of the present invention. For example, the target biomolecules can be albumin, creatinine, or Ngal as well. Besides, the sensing circuit may or may not include a processor to process the measurements. For example, the measurements may be outputted to an external processor (not shown) to determine the concentration of the biomolecules and then have the result back. It should be noted that those skilled in the art can make modification without departing from the spirit of the present invention.

In detail, the sensing chip 100 includes a substrate 110 and an antibody layer 120 disposed on the substrate 110. The substrate may comprise glass, silicon, print circuit board, or polymer. The antibodies on the antibody layer 120 are specific to the target biomolecules (hemoglobin or glycosylated hemoglobin) and are meant to capture the target biomolecules. Please note that, in accordance with an example of the present invention, a number of layers of elements may be disposed between the substrate 110 and the antibody layer 120 to increase the bonding strength between the antibodies and the substrate 110. For example, between the substrate 110 and the antibody layer 120, a layer of chromium can be inserted, or moreover a layer of gold can further be inserted on the layer of chromium. The antibody layer 120 can be attached to the surface of the layer of gold. The layer of chromium can enhance the bonding of the gold to a glass substrate. It will be appreciated by those skilled in the art that chromium can be replaced with other types of elements that enhances the binding of the layer of gold and the substrate 110. Alternatively, the layer of chromium can be omitted, and the layer of gold may be deposited directly onto the substrate 100. However, it is for illustrative purpose only, and not meant to be a limitation of the present invention.

In addition, please refer to FIG. 2, which illustrates a schematic diagram of an electro-immuno sensing device 20 in accordance with a first embodiment of the present invention. As illustrated in FIG. 2, the electro-immuno sensing device 10 has a compartment 500. The compartment 500 may contain liquid, such as double distilled water. The test sample 900 can be deposited in the compartment 500. When the test sample 900 is in contact with the liquid in the sensing chip 100, the membranes of the blood cells in the test sample 900 will break, and release the target biomolecules (e.g., hemoglobin or glycosylated hemoglobin). In this embodiment, the sensing circuit 220 includes a pair of electrodes 222, 224. The electrodes 222, 224 may be any conductive elements, such as copper, tin, silver and gold. The sensing chip 100 includes a pair of sensing pieces 140, 150. The sensing pieces 140, 150 have antibodies disposed thereon such that when the test sample 900 is deposited in the compartment 500, the antibodies on the sensing pieces 140, 150 may bind the target biomolecules in the test sample 900. The signal generator 210 is electrically coupled to the electrodes 222, 224 of the sensing circuit 220 and can continuously generate the signal SIG. The sensing circuit 220 measures an electrical characteristic, such as capacitance, impedance, inductance or phase angle between the electrodes 222, 224, and determines the concentration of the target biomolecules in a test sample 900. Please note that, the number of sensing pieces and the relative positions of the sensing pieces 140, 150 regarding the electrodes 222, 224 are for illustrative purpose only, and not meant to be a limitation of the present invention. For example, in this embodiment, the sensing pieces 140, 150 are parallel with the electrodes 222, 224 and are disposed at the same level of the electrodes 222, 224. In another embodiment, please refer to FIG. 3, which is a schematic diagram of an electro-immuno sensing device 30 in accordance with a second embodiment of the present invention. In FIG. 3, the sensing pieces 140, 150 are still parallel with the electrodes 222, 224, but are disposed below the electrodes 222, 224. In yet another embodiment, please refer to FIG. 4, which is a schematic diagram of an electro-immuno sensing device 40 in accordance with a third embodiment of the present invention. In FIG. 4, there is only one sensing pieces 140, and the sensing pieces 140 are vertical to the electrodes 222, 224. In yet another embodiment, please refer to FIG. 5, which is a schematic diagram of an electro-immuno sensing device 50 in accordance with a fourth embodiment of the present invention. In FIG. 5, the sensing chip 100 is further integrated in to the electrodes of the sensing circuit 220. That is, the antibodies which are originally meant to be disposed on the sensing chip 100, are disposed on the electrodes of the sensing circuit 220. In other words, the electrodes of the sensing circuit 220 is used as the substrate 110 of the sensing chip 100.

In short, as long as the sensing chip 100 is configured in the electrical field that generates the electronic signal SIG, those skilled in their art can make modifications and alternation of the number of sensing pieces of the sensing chip 100 and the relative positions of the sensing chip 100 regarding the electrodes 222, 224 of the sensing circuit 220 without departing from the spirit of the present invention.

For example, please refer to FIG. 6, which a schematic diagram of an electro-immuno sensing device 60 in accordance with a fifth embodiment of the present invention. The electro-immuno sensing device 60 is similar to the electro-immuno sensing device 20. The main difference is that the electro-immuno sensing device 10 has another compartment 600, the sensing chip 100 has other pair of sensing pieces 160, 170, and the sensing circuit 220 has another pair of electrodes 226, 228. The compartments 500, 600 may contain liquid, such as double distilled water. The test sample 900 can be deposited in the compartments 500, 600. When the test sample 900 is in contact with the liquid in the sensing chip 100, the membranes of the blood cells in the test sample 900 will break, and release the target biomolecules (e.g., hemoglobin or glycosylated hemoglobin). The sensing pieces 140, 150 have antibodies disposed thereon while the sensing pieces 160, 170 have no antibodies. When the test sample 900 is deposited in the compartment 500, 600, the antibodies on the sensing pieces 140, 150 may bind the target biomolecules in the test sample 900. The signal generator 210 is electrically coupled to the electrodes 222, 224, 226, 228 of the sensing circuit 220 and can continuously generates the signal SIG. The sensing circuit 220 measures electrical characteristics, such as capacitance, impedance, inductance or phase angle, between the electrodes 222, 224 and between the electrodes 226, 228. The sensing circuit 220 determines the concentration of the target biomolecules in a test sample 900 according to the difference the electrical characteristics between the electrodes 222, 224 and between the electrodes 226, 228.

In addition, please refer to FIG. 7, which is a schematic diagram of an electro-immuno sensing device 70 in accordance with a sixth embodiment of the present invention. The electro-immuno sensing device 70 is similar to the electro-immuno sensing device 60. The main difference is that the electro-immuno sensing device 70 employs a mixing unit 700 to mix the test sample 900 with the liquid contained in the compartment 500, 600. In this embodiment, the mixing unit 700 includes a pair of stirring motors 712, 714 and a pair of stirrers 722, 724. The stirrers 722, 724 are placed in the compartments 500, 600, respective. The stirring motors 712, 714 may be turned on to cause the stirrers 722, 724 in the compartments 500, 600 to spin at a predetermined speed for a predetermined amount of time, respective, such that the target biomolecules (e.g., hemoglobin or glycosylated hemoglobin) which may have fallen at the bottom of the compartments 500, 600 can be propelled upward toward the sensing pieces 160, 170, 140, 150.

Please refer to FIG. 8, which is a schematic diagram of an electro-immuno sensing device 80 in accordance with a seventh embodiment of the present invention. The electro-immuno sensing device 80 is similar to the electro-immuno sensing device 70. The main difference is that the mixing unit 800 includes a motor 801, a shaft 802 and a cam 803. The motor 801 rotates the shaft 802, and the cam 803 on the shaft causes the mixing unit 800 to vibrate. The mixing unit 800 can be in contact with the compartments 500, 600, such that the vibration generated by the mixing unit 800 can cause fluid to flow in the compartment s 500, 600.

Please refer to FIG. 9, which is a schematic diagram of an electro-immuno sensing device 90 in accordance with an eighth embodiment of the present invention. The electro-immuno sensing device 90 is similar to the electro-immuno sensing device 70. The main difference is that the mixing unit 900 includes a rotation means 901 where the compartments 500, 600 are inserted. An exemplary rotation means 901 in accordance with the present invention can comprise a gear 910, and the rotation means 901 can be configured to rotate back and forth, such that the fluid in the compartments 500, 600 can thus be mixed.

The concept of the invention is to measure a variation of the electrical characteristic such as capacitance, impedance, inductance or phase angle, of a first state and a second state. The first state is different to the second state due to immobilization of the target biomolecules. If the test sample 900 contains the target biomolecules, the concentration of the target biomolecules will change and thus the measured electrical characteristic will change accordingly. Therefore, the percentage of target biomolecules can be determined. Take the embodiment in FIG. 6 for example, the first state may be that there is no antibodies but the target biomolecules in the solution, and the second state is that the antibodies have bound with the target biomolecules and there are target biomolecules in the solution; in the embodiments in FIG. 3-5, the first state may be that there is antibodies but no target biomolecules in the solution, and the second state may be that the antibodies have bound with the target biomolecules and there are target biomolecules in the solution; or the first state may be that there are antibodies but no target biomolecules in the solution, and the second state may be that the antibodies have bound with the target biomolecules and there are no target biomolecules in the solution; or the first state may be that the antibodies have bound with the target biomolecules and there are target biomolecules in the solution, and the second state may be that the antibodies have bound with the target biomolecules and there are no target biomolecules in the solution.

In order to quantify the amount of hemoglobin or glycosylated hemoglobin in blood sample based on measured resistances and capacitances, experiments for finding the correlation between measured resistance and hemoglobin concentration in blood and the correlation between measured capacitance and hemoglobin concentration in blood are designed and performed at least in the following examples.

In one experiment, 31 blood samples were prepared. The hemoglobin concentration of each sample was measured using a Hemocue® Analyzer by Sweden. Subsequently, for each blood sample, a 4 microliter (μL) blood sample was added to the sensing chip 100 containing 396 μL of double distilled water. The sensing pieces 140, 150 were coated with hemoglobin-specific antibodies. The electronic test unit 200 generates an AC signal at 200 kHz, and continuously measures the parallel resistance, series resistance and series capacitance of the liquid between the first and second electrodes 222, 224, from a time before the blood sample was added to the sensing chip 100, until the time the measurements shows that the binding of hemoglobin and antibody has reached an equilibrium.

The measurements obtained at equilibriums were plotted against the hemoglobin concentration measured by the Hemocue® Analyzer. FIG. 10 illustrates a graph of parallel resistance vs. hemoglobin concentration, FIG. 11 illustrates a graph of series resistance vs. hemoglobin concentration, FIG. 12 illustrates a graph of series capacitance vs. hemoglobin concentration and FIG. 13 illustrates a graph of series resistance vs. glycosylated hemoglobin (i.e, Hb A1C) concentration. The coefficient of determination of the data in each graph is close to 1, which shows that the linear correlation between the measured parallel resistance, series resistance and series capacitance and the hemoglobin concentration is high.

The correlation between measured parallel resistance, series resistance or series capacitance, and glycosylated hemoglobin concentration can be obtained in a similar manner. Therefore, the present invention can determine the percentage of glycosylated hemoglobin in blood samples based on resistance or capacitance measured across a container containing a blood sample.

It will be appreciated by those skilled in the art that changes could be made to the examples described above without departing from the broad inventive concept thereof. It is understood, therefore, that this invention is not limited to the particular examples disclosed, but it is intended to cover modifications within the spirit and scope of the present invention as defined by the appended claims.

Further, in describing representative examples of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. An electro-immuno sensor, comprising:
a signal generator, for generating an electronic signal having a predetermined frequency and a waveform;
a sensing chip, disposed in an electrical field that generates the electronic signal, for capturing target biomolecules in a solution using antibodies; and
a sensing circuit, having a first electrode and a second electrode in contact with the solution, for measuring an electrical characteristic of a first state and the electrical characteristic of a second state between the first and second electrodes, due to the capture of the target biomolecules, and determining the concentration of the target biomolecules according to the variation of the electrical characteristic.

2. The electro-immuno sensor of claim 1, wherein the first state is that there is no antibody but the target biomolecules in the solution, and the second state is that the antibodies have bound with the target biomolecules and there are target biomolecules in the solution.

3. The electro-immuno sensor of claim 1, wherein the first state is that there are antibodies but no target biomolecules in the solution, and the second state is that the antibodies have bound with the target biomolecules and there are target biomolecules in the solution.

4. The electro-immuno sensor of claim 1, wherein the first state is that there are antibodies but no target biomolecules in the solution, and the second state is that the antibodies have bound with the target biomolecules and there are no target biomolecules in the solution.

5. The electro-immuno sensor of claim 1, wherein the first state is that the antibodies have bound with the target biomolecules and there are target biomolecules in the solution, and the second state is that the antibodies have bound with the target biomolecules and there are no target biomolecules in the solution.

6. The electro-immuno sensor of claim 1, wherein the frequency of the electronic signal is in between 0.5 hertz and 2 megahertz.

7. The electro-immuno sensor of claim 1, wherein the waveform of the electronic signal is a sine wave, a square wave, a triangular wave or a saw-tooth wave.

8. The electro-immuno sensor of claim 1, wherein the sensing chip is vertical to or parallel with the first electrode and the second electrode.

9. The electro-immuno sensor of claim 1, wherein the electrical characteristic is a capacitance, impedance, inductance or phase angle.

10. The electro-immuno sensor of claim 1, wherein the sensing chip comprises:
a substrate; and
an antibody layer, disposed on the substrate, for capturing the target biomolecules in the solution.

11. The electro-immuno sensor of claim 10, wherein the substrate is the first electrode.

12. The electro-immuno sensor of claim 10, wherein the substrate comprises glass, silicon, print circuit board, or polymer.

13. The electro-immuno sensor of claim 10, wherein antibodies on the antibody layer are specific to the target biomolecules.

14. The electro-immuno sensor of claim 10, wherein the sensing chip further comprises:
a gold layer, disposed in between the substrate and the antibody layer, for increasing the immobilization of the antibody layer on the substrate.

15. The electro-immuno sensor of claim 14, wherein the sensing chip further comprises:
a chromium layer, disposed in between the substrate and the gold layer, for increasing the evaporation of the gold layer on the substrate.

16. The electro-immuno sensor of claim 1, wherein the target biomolecule is hemoglobin or glycosylated hemoglobin, albumin, creatinine, or Ngal.

17. The electro-immuno sensor of claim 1, further comprising:
a mixing unit, for mixing a test sample with the solution.

18. The electro-immuno sensor of claim 17, wherein the mixing unit comprises:
a stirring motor, for causing a stirrer in the solution to spin.

19. The electro-immuno sensor of claim 17, wherein the mixing unit comprises:
a vibrator, for causing the solution to vibrate.

20. The electro-immuno sensor of claim 17, wherein the mixing unit comprises:
a rotating device, for turning a compartment which accommodates the solution upside down back and forth.
